(19) United States
(12) Patent Application Publication
(10) Pub. No.: US 2018/0207268 A1
(43) Pub. Date: Jul. 26, 2018

(54) COMBINATION THERAPY FOR CANCER

(71) Applicant: ImClone LLC, Indianapolis, IN (US)

(72) Inventors: Sudhakar R. CHINTHARLAPALLI, Zionsville, IN (US); Anthony S. FISCHL, Carmel, IN (US); Victoria Lynn PEEK, Noblesville, IN (US); Richard A. WALGREN, New Palestine, IN (US); Sau Chi Betty YAN, Indianapolis, IN (US)

(73) Assignee: ImClone LLC, Indianapolis, IN (US)

(21) Appl. No.: 15/745,451

(22) PCT Filed: Aug. 10, 2016
(86) PCT No.: PCT/US2016/046259

§ 371 Date: Jan. 17, 2018

Related U.S. Application Data
(60) Provisional application No. 62/204,088, filed on Aug. 12, 2015.

(51) Int. Cl. A61K 39/395; A61K 31/4439; A61K 45/06; A61P 35/00; C07K 16/28; C07K 16/30

(52) U.S. Cl. CPC A61K 39/39558; A61K 31/4439; A61K 45/06; A61K 2039/505; C07K 16/2863; C07K 16/3046; A61K 2300/00; A61P 35/00

(57) ABSTRACT

The present invention relates to a combination of an anti-human VEGFR2 antibody, preferably ramucirumab, and N-(3-fluoro-4-(1-methyl-6-(1H-pyrazol-4-yl)-1H-indazol-5-yloxy)phenyl)-1-(4-fluorophenyl)-6-methyl-2-oxo-1,2- dihydropyridine-3-carboxamide, and to methods of using the combination to treat certain disorders, such as gastric cancer.

The present invention relates to a combination of an anti-human VEGFR2 antibody, preferably ramucirumab, and N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, and to methods of using the combination to treat certain disorders, such as gastric cancer.

The present invention is in the field of treatment of gastric cancer.

MKN45 gastric cancer cells have a high level of genomic amplification of MET, which leads to the constitutive activation of the MET pathway.

Ramucirumab (Cyramza®) is a fully human monoclonal antibody directed against the vascular endothelial growth factor receptor 2 (VEGFR2). Ramucirumab and methods of making and using this compound including for the treatment of neoplastic diseases such as solid and non-solid tumors are disclosed in WO2003/075840. Ramucirumab is approved by the U.S. F.D.A. as a single agent, or in combination with paclitaxel, for the treatment of patients with advanced or metastatic gastric or gastroesophageal (GE) junction adenocarcinoma with disease progression on or after prior fluoropyrimidine- or platinum-containing chemotherapy; in combination with docetaxel, for the treatment of patients with metastatic non-small cell lung cancer (NSCLC) with disease progression on or after platinum-based chemotherapy; and in combination with FOLFIRI (irinotecan, folinic acid, and 5-fluorouracil) chemotherapy, for the treatment of patients with metastatic colorectal cancer (mCRC) with disease progression on or after prior therapy with bevacizumab, oxaliplatin, and a fluoropyrimidine.

N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is active against MET. N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide and methods of making and using this compound including for the treatment of cancer and more specifically for the treatment of gastric cancer are disclosed in WO2010/011538. Furthermore, N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is currently being investigated in a Phase I clinical trial for advanced cancer in the United States (A Phase 1 Study of LY2801653 in Patients With Advanced Cancer, NCT01285037). The objective of one portion of the study is to determine a recommended Phase 2 dose of LY2801653 that may be safely given to participants with gastric cancer when taken with ramucirumab. (https://clinicaltrials.gov/ct2/show/NCT01285037?term=LY2801653&rank=2).

A cure for gastric cancer still remains elusive and there exists a need for more and different therapies that may prove to be effective in treating gastric cancer.

Although combinations of inhibitors of VEGFR2 and MET inhibitors have been contemplated in the art, surprisingly, the present invention discloses methods of treating gastric cancer by using a combination of an anti-VEGFR2 Ab and N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide as part of a specific treatment regimen that provides enhanced and/or unexpected beneficial therapeutic effects from the combined activity of these therapeutic agents as compared to the therapeutic effects provided by either agent alone.

According to the first aspect of the present invention, there is presented a method of treating gastric cancer in a patient, comprising administering to a gastric cancer patient in need of such treatment an effective amount of an antibody comprising a light chain variable region (LCVR) amino acid sequence of SEQ ID NO: 1 and a heavy chain variable region (HCVR) amino acid sequence of SEQ ID NO: 2, wherein the antibody binds to VEGFR2, and a compound of the formula:

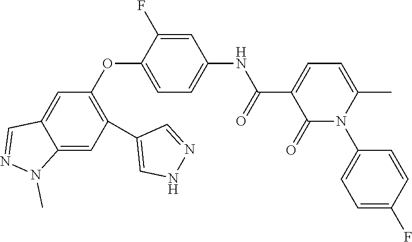 [SMILES: Cc1ccc(C(=O)Nc2ccc(Oc3cc4cnn(C)c4cc3-c3cn[nH]c3)c(F)c2)c(=O)n1-c1ccc(F)cc1]

or a pharmaceutically acceptable salt thereof.

Another aspect of the invention is a kit comprising an antibody comprising a light chain variable region (LCVR) amino acid sequence of SEQ ID NO: 1, and a heavy chain variable region (HCVR) amino acid sequence of SEQ ID NO: 2, wherein the antibody binds to VEGFR2, and a compound of the formula:

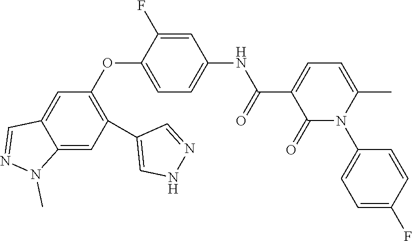 [SMILES: Cc1ccc(C(=O)Nc2ccc(Oc3cc4cnn(C)c4cc3-c3cn[nH]c3)c(F)c2)c(=O)n1-c1ccc(F)cc1]

or a pharmaceutically acceptable salt thereof, for the treatment of gastric cancer.

Another aspect of the invention is a kit, comprising ramucirumab, with one or more pharmaceutically acceptable carriers, diluents, or excipients, and N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, with one or more pharmaceutically acceptable carriers, diluents, or excipients, for the treatment of gastric cancer.

In a preferred aspect of the invention, the compound is N-(3-fluoro-4-(1-methyl-6-(1H-pyrazol-4-yl)-1H-indazol-5-yloxy)phenyl)-1-(4-fluorophenyl)-6-methyl 2-oxo-1,2-dihydropyridine-3-carboxamide.

In another preferred aspect of the invention, the antibody comprises a light chain amino acid sequence of SEQ ID NO: 3, and a heavy chain amino acid sequence of SEQ ID NO: 4 and the antibody binds to VEGFR2.

In another preferred aspect of the invention, the antibody is ramucirumab.

In another preferred aspect of the invention, the compound or salt thereof is administered at a dose of between about 80 mg/day to about 120 mg/day.

In another preferred aspect of the invention, ramucirumab is administered once every three weeks at a dose of between about 6 mg/kg to about 12 mg/kg.

In another preferred aspect of the invention N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, is a tablet.

In another preferred aspect of the invention, the tablet is formulated by Spray Dried Dispersion.

Another aspect of the invention is a combination comprising an anti-VEGFR2 antibody, preferably ramucirumab, and N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, for simultaneous, separate or sequential use in the treatment of gastric cancer. In preferred aspect of the invention, the compound or salt thereof is administered at a dose of between about 80 mg/day to about 120 mg/day. In another preferred aspect of the invention, ramucirumab is administered once every three weeks at a dose of between about 6 mg/kg to about 12 mg/kg.

Another aspect of the invention is an anti-VEGFR2 antibody for use in simultaneous, separate or sequential treatment with N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, for the treatment of gastric cancer.

Another aspect of the invention is N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, for use in simultaneous, separate or sequential treatment with an anti-VEGFR2 antibody, for the treatment of gastric cancer.

In another preferred aspect of the invention the anti-VEGFR2 antibody is ramucirumab.

Another aspect of the invention is ramucirumab for use in simultaneous, separate or sequential treatment with N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, for the treatment of gastric cancer.

Another aspect of the invention is N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, for use in simultaneous, separate or sequential treatment with ramucirumab, for the treatment of gastric cancer.

Another aspect of the invention is the use of an anti-VEGFR2 antibody in the manufacture of a medicament for the treatment of gastric cancer wherein the anti-VEGFR2 antibody is administered simultaneously, separately or sequentially with N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof.

Another aspect of the invention is the use of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, in the manufacture of a medicament for the treatment of gastric cancer wherein N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, is administered simultaneously, separately or sequentially with an anti-VEGFR2 antibody.

In yet another preferred aspect of the invention the anti-VEGFR2 antibody is ramucirumab.

Another aspect of the invention is an anti-VEGFR2 antibody for simultaneous, separate or sequential use in combination with N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, for the treatment of gastric cancer.

Another aspect of the invention is N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, for simultaneous, separate or sequential use in combination with an anti-VEGFR2 antibody, for the treatment of gastric cancer.

As used herein, the compound, N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, is disclosed in WO2010/011538 and refers to the compound with the following structure:

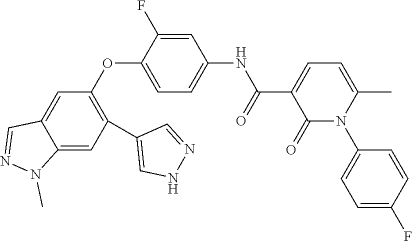 [SMILES: Cc1ccc(C(=O)Nc2ccc(Oc3cc4cnn(C)c4cc3-c3cn[nH]c3)c(F)c2)c(=O)n1-c1ccc(F)cc1]

This compound's CAS registry number is 1206799-15-6. Compound names include: 3-Pyridinecarboxamide, N-[3-fluoro-4-[[1-methyl-6-(1H-pyrazol-4-yl)-1H-indazol-5-yl]oxy]phenyl]-1-(4-fluorophenyl)-1,2-dihydro-6-methyl-2-oxo-; N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide; N-(3-fluoro-4-{[1-methyl-6-(1H-pyrazol-4-yl)-1H-indazol-5-yl]oxy}phenyl)-1-(4-fluorophenyl)-6-methyl-2-oxo-1,2-dihydropyridine-3-carboxamide; and LY2801653.

Metabolites of LY2801653 include:

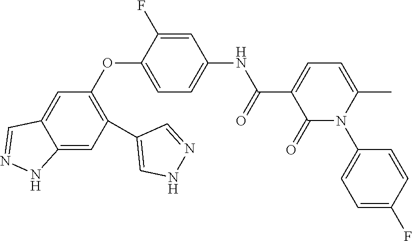 [SMILES: Cc1ccc(C(=O)Nc2ccc(Oc3cc4cn[nH]c4cc3-c3cn[nH]c3)c(F)c2)c(=O)n1-c1ccc(F)cc1]

1-(4-fluorophenyl)-N-[3-fluoro-4-[[6-(1H-pyrazol-4-yl)-1H-indazol-5-yl]oxy]phenyl]-6-methyl-2-oxo-pyridine-3-carboxamide, and,

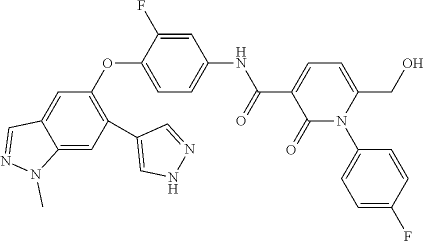 [SMILES: Cn1ncc2cc(Oc3ccc(NC(=O)c4ccc(CO)n(-c5ccc(F)cc5)c4=O)cc3F)c(-c3cn[nH]c3)cc21]

N-[3-fluoro-4-[1-methyl-6-(1H-pyrazol-4-yl)indazol-5-yl]oxy-phenyl]-1-(4-fluorophenyl)-6-(hydroxymethyl)-2-oxo-pyridine-3-carboxamide.

As used herein, the term “VEGFR2” refers to Vascular Endothelial Growth Factor Receptor 2, which is known in the art. VEGFR2 is also known as KDR.

As used herein, the term “anti-VEGFR2 Ab” refers to an antibody comprising: a light chain variable region (LCVR) whose amino acid sequence is that given in SEQ ID NO: 1, and a heavy chain variable region (HCVR) whose amino acid sequence is that given in SEQ ID NO: 2, wherein the anti-VEGFR2 Ab binds to VEGFR2 with sufficient affinity and specificity. In some embodiments, an anti-VEGFR2 Ab is an antibody comprising: a light chain whose amino acid sequence is that given in SEQ ID NO: 3, and a heavy chain whose amino acid sequence is that given in SEQ ID NO: 4 and that binds to VEGFR2 with sufficient affinity and specificity. In other embodiments of the present invention the anti-VEGFR2 Ab is ramucirumab. The antibody selected will have a sufficiently strong binding affinity for VEGFR2. For example, the antibody will generally bind VEGFR2 with a K_{d} value of between about 100 nM-about 1 pM. Antibody affinities may be determined by a surface plasmon resonance based assay (such as the BIAcore assay is described in PCT Application Publication No. WO2005/012359); enzyme-linked immunosorbent assay (ELISA); and competition assays (e.g. a radiolabeled antigen binding assay (RIA)), for example. In one embodiment, Kd is measured by a RIA performed with an anti-VEGFR2 Ab, preferably ramucirumab.

As used herein, the term “ramucirumab” also known as Cyramza®, IMC-1121b, CAS registry number 947687-13-0, refers to an anti-VEGFR2 Ab comprising: two light chains, each of whose amino acid sequence is that given in SEQ ID NO: 3, and two heavy chains, each of whose amino acid sequence is that given in SEQ ID NO: 4.

As used herein, the term “DC101” refers to a rat monoclonal antibody directed against mouse VEGFR2 that may be used in experiments as a surrogate in mice for an anti-VEGFR2 Ab, preferably ramucirumab. See, for example, Witte L., et al Cancer Metastasis Rev., 17, 155-161, 1998.

Unless indicated otherwise, the term “antibody” refers to an immunoglobulin molecule comprising two heavy chains (HC) and two light chains (LC) interconnected by disulfide bonds. The amino terminal portion of each chain includes a variable region of about 100 to about 110 amino acids primarily responsible for antigen recognition via the complementarity determining regions (CDRs) contained therein. The carboxy-terminal portion of each chain defines a constant region primarily responsible for effector function.

As used herein, the term “light chain variable region” or “LCVR” refers to a portion of a light chain of an antibody molecule that includes amino acid sequences of CDRs and FRs.

As used herein, the term “heavy chain variable region” “HCVR” refers to a portion of a heavy chain of an antibody molecule that includes amino acid sequences of CDRs and FRs.

As used herein, the term “kit” refers to a package comprising at least two separate containers, wherein a first container contains N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, and a second container contains an anti-VEGFR2 Ab. A “kit” may also include instructions to administer all or a portion of the contents of these first and second containers to a cancer patient, preferably a gastric cancer patient.

As used herein, the terms “treating,” “to treat,” or “treatment” refers to restraining, slowing, stopping, reducing, shrinking, maintaining stable disease, or reversing the progression or severity of an existing symptom, disorder, condition, or disease.

As used herein, the term “patient” refers to a mammal, preferably a human.

As used herein, the terms “cancer” and “cancerous” refer to or describe the physiological condition in patients that is typically characterized by unregulated cell proliferation. Included in this definition are benign and malignant cancers. By “early stage cancer” or “early stage tumor” is meant a cancer that is not advanced or metastatic or is classified as a Stage 0, 1, or II cancer. Examples of cancer include, but are not limited to, gastric cancer.

A main advantage of the combination treatments of the invention is the ability of producing marked anti-cancer effects in a patient without causing significant toxicities or adverse effects, so that the patient benefits from the combination treatment method overall. The efficacy of the combination treatment of the invention can be measured by various endpoints commonly used in evaluating cancer treatments, including but not limited to, tumor regression, tumor weight or size shrinkage, time to progression, overall survival, progression free survival, overall response rate, duration of response, and quality of life. The therapeutic agents used in the invention may cause inhibition of metastatic spread without shrinkage of the primary tumor, may induce shrinkage of the primary tumor, or may simply exert a tumoristatic effect. Because the invention relates to the use of a combination of unique anti-tumor agents, novel approaches to determining efficacy of any particular combination therapy of the present invention can be optionally employed, including, for example, measurement of plasma or urinary markers of angiogenesis and measurement of response through radiological imaging.

As used herein, the term “effective amount” refers to the amount or dose of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof and the amount or dose of an anti-VEGFR2 Ab which provides an effective response in the patient under diagnosis or treatment.

As used herein, the term “effective response” of a patient or a patient's “responsiveness” to treatment with a combination of agents refers to the clinical or therapeutic benefit imparted to a patient upon administration of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide or a pharmaceutically acceptable salt thereof and an anti-VEGFR2 Ab.

Dosages per day of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide or a pharmaceutically acceptable salt thereof normally fall within the range of about 80 mg/day to 120 mg/day.

Dosages of ramucirumab per three-week cycle normally fall within the range of about 6 to 12 mg/kg, preferably about 8 to about 10 mg/kg, and most preferably about 10 mg/kg.

When given in combination with an anti-VEGFR2 Ab, for example, over a 21-day cycle, N-(3-fluoro-4-(1-methyl-6-(H-pyrazol-4-yl)-1H-indazol-5-yloxy)phenyl)-1-(4-fluorophenyl)-6-methyl-2-oxo-1,2-dihydropyridine-3-carboxamide or a pharmaceutically acceptable salt thereof is administered daily within the range of about 80 mg/day to about 120 mg/day and an anti-VEGFR2 Ab, preferably ramucirumab, is administered on day one within the range of about 6 to 12 mg/kg.

The free base, N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, is preferred. However, it will be understood by the skilled reader that N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide can react with any of a number of inorganic and organic acids to form pharmaceutically acceptable acid addition salts. Such pharmaceutically acceptable acid addition salts and common methodology for preparing them are well known in the art. See, e.g., P. Stahl, et al., Handbook of Pharmaceutical Salts: Properties, Selection and Use (VCHA/Wiley-VCH, 2002); L. D. Bighley, et al., Encyclopedia of Pharmaceutical Technology, 453-499 (1995); S. M. Berge, et al., Journal of Pharmaceutical Sciences, 66, 1, (1977). The hydrochloride and mesylate salts are preferred salts. The mesylate salt is an especially preferred salt.

N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide or pharmaceutically acceptable salts thereof, may be prepared by a variety of procedures known in the art (e.g., see WO2010/011538). Ramucirumab can be made, for example, according to the disclosure in WO2003/075840.

The route of administration may be varied in any way, limited by the physical properties of the drugs and the convenience of the patient and the caregiver. Preferably, an anti-VEGFR2 Ab, preferably ramucirumab, is formulated for parenteral administration, such as intravenous or subcutaneous administration. Preferably, N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or pharmaceutically acceptable salt thereof; is formulated for oral or parenteral administration, including intravenous or subcutaneous administration.

For example, N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide may be formulated into a tablet. Such tablet can be made from a composition of 20% N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide:Hydroxy Propyl Methyl Cellulose Acetate Succinate (HPMCAS) Medium Grade (M) (HPMCAS-M) Spray Dried Dispersion (SDD). The 20% N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide:HPMCAS-M SDD is made from a spray solution composition (wt %) containing N-(3-fluoro-4-(-methyl-6-(1H-pyrazol-4-yl)-1H-indazol-5-yloxy)phenyl)-1-(4-fluorophenyl)-6-methyl-2-oxo-1,2-dihydropyridine-3-carboxamide (1%), HPMCAS-M (4%) and Acetone (85.5%) and purified water (9.5%). Ensure N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is fully solubilized in the acetone/water solution before addition of the polymer. Before initiating spray drying to make the SDD composition, visually confirm that the polymer is dissolved. The resulting SDD composition is a 20% N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide:HPMCAS-M SDD (mg/g) with N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide (200 mg/g) and HPMCAS-M (800 mg/g). If necessary, the amount of drug substance may be adjusted to take into account the assay of the drug substance. If required to maintain mass balance, the weight of HPMCAS-M may be adjusted according to slight changes in assay of the drug substance. Acetone and purified water are removed during processing to residual levels. The formulation composition can contain, for example, SDD N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide and other excipients such as diluent (e.g. microcrystalline cellulose and mannitol), disintegrant (e.g. croscarmellose sodium), surfactant (e.g. sodium lauryl sulphate), glidant (e.g. syloid silicon dioxide) and/or lubricant (e.g. sodium stearyl fumarate). The making of the tablet involves spray drying to produce the SDD of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide followed by roller compaction and compression into tablets. The tablets are then film-coated with HPMC based color mixture.

An example tablet for a unit formula of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide SDD film coated tablet, 40 mg dose strength, is described in Chart 1.

### Chart 1

### Example Unit Formula of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide

Tablets 40 mg

| | | |
|---|---|---|
| | | mg/ |
| Ingredient | Function | Tablet |

Active Ingredient

| | | |
|---|---|---|
| 20% N-(3-fluoro-4-(1-methyl-6-(1H-pyrazol- | Active | 200 |
| 4-yl)-1H-indazol-5-yloxy)phenyl)-1-(4- | | |
| fluorophenyl)-6-methyl-2-oxo-1,2-dihydro- | | |
| pyridine-3-carboxamide: 80% HPMCAS-M SDD¹ | | |

Intragranular Excipients²

| | | |
|---|---|---|
| Microcrystalline Cellulose (Avicel PH 102 ®)³ | Filler | 20.19 |
| Mannitol (Pearlitol S D 200 ®) | Filler | 19.50 |
| Croscarmellose Sodium | Disintegrant | 6.50 |
| Sodium Lauryl Sulphate | Surfactant | 3.250 |
| Silicon Dioxide (Syloid 244FP ®) | Gildant | 3.250 |
| Sodium Stearyl Fumarate | Lubricant | 2.440 |

Extragranular Excipients

| | | |
|---|---|---|
| Microcrystalline Cellulose (Avicel PH 102 ®) | Filler | 65.00 |
| Croscarmellose Sodium | Disintegrant | 3.250 |
| Sodium Stearyl Fumarate | Lubricant | 1.620 |

| | |
|---|---|
| Total Core Tablet Weight (mg) | 325.0 |

Film Coating Excipients

| | | |
|---|---|---|
| Color Mixture Blue 03K105008 | Film Coat | 11.38 |
| Purified Water⁴ | Suspension | — |
| | Vehicle | |

| | |
|---|---|
| Total Coated Tablet Weight (mg) | 336.4 |

¹If necessary, the amount of SDD will be adjusted to take into account of the assay of the dispersion.
²A reasonable variation of ±10% is allowed for each excipient unless otherwise stated.
³To accommodate changes in SDD potency and to maintain the total tablet weight, the weight of microcrystalline cellulose may be adjusted if necessary.
⁴Purified water is removed during processing to residual levels.

As used herein, the phrase “in combination with” refers to the administration of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, and an anti-VEGFR2 Ab.

The following examples illustrate the unexpected improvement of the combination of an anti-VEGFR2 Ab, including, but not limited to, ramucirumab, (via the rat anti-mouse monoclonal antibody used in mice as a surrogate for ramucirumab, DC101) and N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide.

### Antitumor Effects of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide in Combination with DC101 in Mouse Xenograft Model MKN45 for Gastric Cancer

N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is formulated as a solution in 10% PEG 400/90% (20% Captisol® in H₂O) and prepared fresh each week of dosing. DC101 is diluted in phosphate-buffered saline (PBS) each week of dosing.

Obtain MKN45 cells and maintain in Dulbecco's Modified Eagle's Medium (DMEM) supplemented with 10% FBS at 37° C. with 5% CO₂. Approximately 5×10⁶ cells in PBS are mixed with an equal volume of Matrigel (BD Bioscience, Franklin Lakes, N.J.) and implanted subcutaneously into the flank of the animal. Animals are randomized into groups of 9 on Day 18 post-implant. N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is administered via oral gavage at 12 mg/kg and DC101 is administered via intraperitoneal injection at 20 mg/kg. Continue dosing for 26 days on a once daily schedule for N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide and on a twice weekly schedule for DC101. A total of 7 doses of DC101 are administered over the course of the study. Tumor volumes and body weight are measured bi-weekly. At the end of the study, animals are sacrificed using CO₂ and cervical dislocation.

Tumor volume is transformed to the log scale to equalize variance across time and treatment groups. The log volume data are analyzed with a two-way repeated measures analysis of variance by time and treatment using the MIXED procedures in SAS® software (Version 9.3). The correlation model for the repeated measures is spatial power. Treated groups are compared to the control group at each timepoint. The MIXED procedure is also used separately for each treatment group to calculate adjusted means and standard errors at each timepoint. Both analyses account for the autocorrelation within each animal and the loss of data that occurs when animals are removed or lost before the end of the study. The adjusted means and standard errors are plotted for each treatment group versus time. These data are also analyzed for statistical evidence (“s.e.”) of an increase in effect over additivity for the combination of two treatments. This analysis is conducted using SAS® software (Version 9.3) by testing for significance of a 2×2 interaction effect on log volume using the vehicle, each single agent, and the combination of each single agent groups. This analysis is used to assess the synergy of effect.

Body weight measurements provide an indication of the tolerability of the various treatments. No statistically significant loss of body weight was observed with treatment. One animal in the vehicle control group was euthanized due to an ulcerated tumor. No treatment-related deaths were reported in this study.

As a monotherapy, N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide demonstrated statistically significant anti-tumor activity, with a T/C (treatment group/control group) value of 4.4% on the final day of measurement (Day 42) (p<0.001). DC101 demonstrated single agent anti-tumor activity with a T/C value of 15.0% (p<0.001). The low T/C value for either agent alone indicated that treatment with either agent alone potently slowed down tumor growth as compared to vehicle control. Animals treated with either of these agents alone achieved the clinical equivalent response of “stable disease” in cancer treatment in this gastric tumor model. Therasse et al. New guidelines to evaluate the response to treatment in solid tumors. J Natl Cancer Inst. 2000; 92:205-216. Eisenhauer et al. New response evaluation criteria in solid tumours: revised RECIST guideline (version 1.1). Eur J Cancer. 2009; 45:228-247.

The combination of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide with DC101 resulted in tumor regression (28.5%) (p<0.001) as compared to the vehicle control, and this activity was greater than either molecule administered as a monotherapy.

The combination of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide with DC101 was additive (Table 1) and statistically different as compared to each monotherapy (Table 2). Thus the treatment of the combination of these two agents in a solid tumor type such as gastric cancer was found to be additive and with further anti-tumor effect than each single agent alone. More importantly, the tumor regression (tumor shrinkage) effect of 28.5% compared to control resulting from the combination treatment of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide with DC101 was unexpected and therapeutically beneficial. The in vivo treatment with the combination of these two agents in a gastric solid tumor model resulted in tumor shrinkage/regression and was therapeutically beneficial.

TABLE 1
Interaction Tests-Tumor Volume for N-(3-fluoro-4-(1-methyl-6-
(1H-pyrazol-4-yl)-1H-indazol-5-yloxy)phenyl)-1-(4-fluorophenyl)-
6-methyl-2-oxo-1,2-dihydropyridine-3-carboxamide and DC101

| | | | % reduction | | Estimate | | |
|---|---|---|---|---|---|---|---|
| | % reduction | % reduction | LY2801653 + | Expected | (log | | |
| Day | LY2801653 | DC101 | DC101 | if Additive | scale) | s.e. | p-value |
| 7 | −4.076 | 13.612 | −0.753 | 10.091 | 0.025 | 0.0441 | 0.577 |
| 11 | −3.942 | 5.119 | 2.517 | 1.379 | −0.003 | 0.0441 | 0.955 |
| 14 | −7.287 | 7.305 | 10.166 | 0.550 | −0.022 | 0.0441 | 0.618 |
| 18 | −8.375 | −1.160 | 1.929 | −9.631 | −0.024 | 0.0441 | 0.585 |
| 20 | 18.997 | 18.490 | 28.749 | 33.974 | 0.017 | 0.0441 | 0.709 |
| 25 | 49.694 | 35.365 | 54.508 | 67.485 | 0.073 | 0.0441 | 0.102 |
| 28 | 58.139 | 41.880 | 62.634 | 75.670 | 0.093 | 0.0441 | 0.038* |
| 32 | 63.836 | 53.652 | 72.374 | 83.239 | 0.109 | 0.0441 | 0.016* |
| 39 | 73.832 | 63.572 | 81.314 | 90.468 | 0.146 | 0.0444 | 0.001* |
| 42 | 75.070 | 66.791 | 84.667 | 91.721 | 0.134 | 0.0445 | 0.004* |

*statistically significant

TABLE 2
Treatment Comparisons-Log Scale on
Final Day of Measurement (Day 42)

| | | | Difference | | |
|---|---|---|---|---|---|
| | | | (Treatment 1 − | | |
| Day | Treatment | Treatment 2 | Treatment 2) | s.e. | p-value |
| 42 | Vehicle | LY2801653 | 0.603 | 0.0634 | <0.001* |
| 42 | Vehicle | DC101 | 0.479 | 0.0634 | <0.001* |
| 42 | Vehicle | DC101/ | 0.814 | 0.0634 | <0.001* |
| | | LY2801653 | | | |
| 42 | LY2801653 | DC101/ | 0.211 | 0.0624 | 0.001* |
| | | LY2801653 | | | |
| 42 | DC101 | DC101/ | 0.336 | 0.0624 | <0.001* |
| | | LY2801653 | | | |

*statistically significant

### Combinations of Ramucirumab and N-(3-fluoro-4-(1-methyl-6-(1H-pyrazol-4-yl)-1H-indazol-5-yloxy)phenyl) 1-(4-fluorophenyl)-6-methyl-2-oxo-1,2-dihydropyridine-3-carboxamide Treatment Reduces Endothelial Cell Sprouting

Measure the in vitro reduction of endothelial cell sprouting by an in vitro cell based assay. Use the assay to measure the effect of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide and ramucirumab on endothelial cell sprouting.

HUVECs (Lonza # C2519A) were cultured at 37° C. in 5% CO₂ on culture flasks (Corning #356486) in EBM2 medium (Lonza # CC-3156) supplemented with SingleQuots kit (Lonza # CC-4147), with further FBS supplement to a final 10% FBS and were used at passages 2-5. HUVECs were harvested from culture flasks which were rinsed with Hyclone™ Dulbecco's PBS (DPBS) (Fisher Scientific, #SH3026402) followed by TypeLE Express (Gibco #12605-010) and were suspended in 5 mL of warm medium. Viable cell counts were determined using a Vi-Cell cell counter (Beckman). CAFs (lung cancer associated fibroblast) (Astrand, 60093A, specially prepared for Lilly) were cultured at 37° C. in 5% CO₂ on Corning culture flasks in FBM medium (Lonza #CC-3131) supplemented with SingleQuots kit (Lonza # CC-4126), with further FBS (Hyclone™ # SH3061102) supplement to a final 10% FBS, and used at passages 3-7. CAFs were harvested from culture flasks which were rinsed with Hyclone™ DPBS (Fisher Scientific, #SH3026402) followed by TypeLE Express (Gibco #12605-010) and were resuspended with 5 ml of warm medium. Viable cell counts were determined using a Vi-Cell cell counter (Beckman).

Dry Cytodex® beads (Sigma-Aldrich®, cat # C3275) of 0.5 g are hydrated in 50 mL HyClone™ DPBS pH 7.4 (Fisher Scientific, cat# SH3026402) for at least 3 hours at room temperature. The tube (Falcon, cat#352098) containing the beads is inverted to mix gently every 0.5 hour. Supernatant is discarded. Beads are washed three times with fresh PBS and re-suspended in 50 mL PBS to give ˜20,000 beads/mL. The bead suspension is autoclaved for 15 minutes at 115° C. and stored at 4° C. until use.

Beads are gently mixed and 0.5 mL (approximately 10,000 beads) suspension is transferred into a 50 mL tube (Falcon, cat#352098). Beads are washed twice in 10 mL of warm EBM2 medium (Lonza cat# CC-3156) plus SingleQuots™ (Lonza cat# CC-4147). The medium is carefully removed after final wash. Washed beads are mixed with 8 million HUVEC cells in total volume of 20 mL. The tube containing beads and HUVEC cells is placed in an incubator at 37° C. with 5% CO₂ for 4 hours and gently mixed every 20 minutes by inverting the tube several times. After incubation, beads with HUVEC cells are transferred into a T25 flask (Nunc™ cat #156499) and incubated at 37° C. with 5% CO₂ overnight.

Fibrinogen (Sigma cat # F4883) is dissolved in HyClone™ DPBS at 2 mg/mL. Aprotinin (Sigma cat # A3428) is added to the fibrinogen solution at a concentration of 0.15 units/mL and gently mixed. The solution is sterilized by filtering through a 0.22μ filter (Millipore #SCGP00525) and used immediately.

The HUVEC-coated beads in the T25 flask is transferred to a 50 mL tube and washed twice using 10 mL of warm EBM2 medium plus SingleQuots (Lonza #CC-4147). The medium is removed gently. HUVEC-coated beads (approximately 10,000) are re-suspended in 50 mL of sterilized fibrinogen solution with 2 million CAFs. Thrombin (Sigma # T4393) was reconstituted with sterile water to 50 units/ml. 0.6 unit (12 μl) of the thrombin solution was added per well of a 24-well plate (In Vitro Scientific cat#P24-1.5H-N), followed by the addition of 500 l/well of the Fibrinogen/beads/CAF solution. The solution is allowed for fibrin gel formation for 15 minutes at room temperature and then at 37° C. with 5% CO₂ for an hour. 0.5 mL of warm EBM2 medium plus SingleQuots (Lonza #CC-4147) is added on top of the fibrin gel in each well and replaced every 3 to 4 days until the end of the experiment.

For the neo-mode sprouting assay, test compound diluted in the indicated concentration is added to each well. Plates are incubated at 37° C. with 5% CO₂ and medium with test compound is changed every 3 to 4 days till the assay is completed.

For the established-mode sprouting assay, the method is the same as described above for the neo-mode sprouting assay, except the HUVEC-coated beads in the fibrin gel are cultured for 3 to 7 days before the addition of the test compound. The test compound treatment goes for 7 days. Medium with test compound is changed every 3 to 4 days till the assay is done.

After the assay is finished, plates are fixed in 0.5 mL of 4% PFA (Electron Microscopy Sciences #15710) overnight at 4° C., washed once in PBS, permeabilized with 0.5 mL of 0.5% Triton™ X-100 (Sigma-Aldrich cat# T9284)/PBS for 10 minutes at 4° C. followed by washing three times in 100 mM glycine (Bio-Rad cat#161-0718)/PBS at room temperature. The plates are blocked with 1 ml/well IF buffer which contains 0.1% BSA (gibco cat#15260-037), 0.2% Triton™ X-100, 0.05% Tween-20 (Thermo Scientific cat #28320) in PBS plus 10% goat serum (Invitrogen #16210). Endothelial cells are stained with sheep anti-human CD31 antibody (R&D Systems #BAF806) reconstituted in 500 mL PBS at 1:100 in IF buffer and 10% goat serum. SMA positive cells are stained with anti-α smooth muscle actin antibody, Cy3 antibody (Sigma, Cat# C6198) at 1:200 in IF buffer plus 10% goat serum. The staining solution is added to each well at 500 μL/well. The plates are kept at 4° C. overnight. The staining solution is removed on the following day, and plates are washed using IF buffer three times, each with 0.5 mL. Secondary antibody Alexa Fluor® 488 Donkey anti-sheep IgG (H+L) (Molecular Probes #A-11015) at 1:200 dilution in the IF buffer plus 10% goat serum is added for an hour incubation at room temperature. The plates are washed three times using IF buffer to remove any unbound secondary antibody. DAPI (4′,6-Diamidino-2-Phenylindole, Dihydrochloride)(Invitrogen #D1306) at 5 mg/mL is diluted at 1:10000 in PBS and 0.5 mL is added to each well for an hour incubation at room temperature. The plates are washed twice with PBS and total length of CD31 positive endothelial sprouts and SMA positive cells are imaged by scanning the plates on a CellInsight (Thermo Scientific) instrument using the 2× Objective. Image data were directly from the CellInsight (CD31, green; SMA, red) and numeric data were analyzed in JMP (SAS).

N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide treatment reduced VEGF-A dependent and VEGF-A independent endothelial sprouting with statistical significance.

A modified in vitro co-culture angiogenesis assay as described in Mabry, R, et al, MAbs. 2010 January-February; 2(1):20-34 and Nakatsu, MN, Meth Enzymol. 2008; 443:65-82) using HUVECs (human umbilical vein endothelial cells) and CAF cells (cultured lung cancer associated fibroblast cells) is used to evaluate the effects of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide on endothelial cell sprouting. CAFs and cytodex beads coated with HUVECs are imbedded into a fibrin gel to form endothelial sprouts that are covered with smooth muscle actin (SMA) positive pericytes. For a one-week period, the endothelial cells undergo a series of phenotypic changes that result in a stable interconnected network of endothelial sprouts that are covered with SMA positive cells. Endothelial sprouting is dependent on CAF derived VEGF-A in the media for up to 7-10 days after which sprout elongation and stability is less dependent on VEGF-A.

Inhibition of VEGF-A signaling at the start of the assay (neo-mode, days 0-7) with ramucirumab (10 μg/ml) led to significant inhibition of endothelial sprouting (4-fold reduction). To mimic therapeutic inhibition of angiogenesis, treatment with ramucirumab is started on day 7 (established mode) after endothelial sprouts and pericyte coverage is formed. Treatment of preformed sprouts with ramucirumab (10 μg/ml) was less effective and only reduced endothelial sprouting 1.4 fold. Similar to VEGFR2 inhibition, addition of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide at the start of the assay (neo-mode, days 0-7) potently inhibited endothelial sprouting in a dose dependent manner. At the highest concentration tested, 300 nM, N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide reduced endothelial sprouting 7.5 fold. Treatment of preformed sprouts (established mode), which are significantly less sensitive to VEGFR2 inhibition, with N-(3-fluoro-4-(1-methyl-6-(H-pyrazol-4-yl)-1H-indazol-5-yloxy)phenyl)-1-(4-fluorophenyl)-6-methyl-2-oxo-1,2-dihydropyridine-3-carboxamide, led to a significant reduction in endothelial sprouting (2.7 fold). These results demonstrated that N-(3-fluoro-4-(1-methyl-6-(H-pyrazol-4-yl)-1H-indazol-5-yloxy)phenyl)-1-(4-fluorophenyl)-6-methyl-2-oxo-1,2-dihydropyridine-3-carboxamide potently inhibits VEGF-A dependent and VEGF-A independent endothelial sprouting. The MET-specific inhibitor PF04217903 (LSN2900296) was less active in inhibiting endothelial sprouting when added at the beginning of the assay (neo-mode) where sprouting is VEGF-A dependent (days 0-7) and when added to preformed sprouts (established mode) that are less dependent on VEGF-A for sprout elongation and stability (days 7-14) PF04217903 was inactive in inhibiting endothelial sprouting. These data suggest the anti-angiogenic activity of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is not MET dependent.

N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide inhibited sprouting through day 14 while ramucirumab inhibited sprouting only up to day 7. Control (PF4217904) had no effect.

Ramucirumab is a specific VEGFR2 inhibitor and as expected demonstrated endothelial sprouting reduction in this assay for the first 7 days when the sprouting is VEGF-A dependent. The MET specific inhibitor PF4217903 showed little or no effect in this assay throughout the 14 days of this assay indicating that MET does not play a role in this vascular model and MET is one of the oncokinase targets for N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide. The data from this assay suggested that one or more of the other targets of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, such as Tie2 (a.k.a. TEK), AXL, PDGFRA, and MERTK was causing inhibition of endothelial sprouting throughout the 14 days of this angiogenesis assay. Thus the anti-angiogenic activity of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is different from ramucirumab and supports the combination of these two agents in cancer treatment to provide further treatment benefit.

### N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide Inhibits Cord Formation in Neo and Established Modes

A VEGF-A induced cord formation assay is performed in micro-titer plates according to Falcon et al., J Hematol Oncol. 2013; 6:31. The assay is performed as the neo-mode (neoangiogenic adipose derived stem cells (“ADSC”) and human endothelial colony forming cells (“ECFC”) co-culture cord formation Assay) and the Established mode (established ADSC and ECFC co-culture cord formation assay).

For the neo-mode assay, the ADSC and ECFC are co-cultured with AngioKit™ optimized media (Cell Systems Biology). ADSC are plated in 96-well plates at 40-50K cells per well in 100 μL and incubated overnight at 37° C., 5% CO₂. The next day, the media is removed and 4-5K ECFC per well in 50-100 μL of media is plated on top of the ADSC monolayer and incubated at 37° C., 5% CO₂ for 3-6 hours before the addition of 20 ng/mL VEGF-A and test compounds. Co-cultures are grown for 7 days, at which time the cells are fixed, stained, and imaged in a scanning device. Cord area is quantified.

For the established mode assay, ADSC and ECFC co-culture are plated as described above for the neo-mode assay. After the ECFC are allowed to attach, 20 ng/mL VEGF-A is used to stimulate and to establish the cord network. On Day 4, the media is changed to contain fresh VEGF-A in the presence or absence of test compound at the indicated concentrations. After addition of the test compound, cultures are allowed to grow an additional 3-4 days before the cells are fixed, stained, and imaged as described above, to investigate network disruption or cord regression.

Ramucirumab has been shown to be effective in the neo-mode of this assay, (IC₅₀=0.48 μg/mL (S.D. 0.30, n=3) [0.48 μg/mL=3.2 nM], but not the established mode (IC₅₀>10 μg/mL). See Falcon et al, J Hematol Oncol. 2013; 6:31. Sunitinib, a small molecule multi-kinase inhibitor with anti-VEGFR2 activity, has been shown to be effective in the neo-mode of this assay (IC₅₀=0.038 μM; S.D. 0.013, n=3), but not the established mode. See Falcon et al, J Hematol Oncol. 2013; 6:31.

N-(3-fluoro-4-(1-methyl-6-(H-pyrazol-4-yl)-1H-indazol-5-yloxy)phenyl)-1-(4-fluorophenyl)-6-methyl-2-oxo-1,2-dihydropyridine-3-carboxamide demonstrated potent activity in both the neo-mode (IC₅₀=0.013 μM; S.D. 0.007, n=5) and the established mode (IC₅₀=0.016 μM 0.011, n=4).

A control MET-specific inhibitor is evaluated in this assay. The control MET-specific inhibitor showed only a slight activity in the neo-mode (IC₅₀=5.61 μM; S.D. 1.80, n=2) and no activity in the established mode (IC₅₀>10 μM; n=2). This suggested that the anti-angiogenic activity of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is not due to its activity for MET.

Ramucirumab is a specific VEGFR2 inhibitor and as expected demonstrated reduction in this cord formation assay in the neo-mode when the cord formation was VEGF-A dependent. The MET specific inhibitor PF4217903 showed little or no effect in this assay both in the neo-mode or in the established mode, indicated that MET does not play a role in this vascular model and MET is one of the oncokinase targets for N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide. The data from this assay suggested that one or more of the other targets of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, such as Tie2 (a.k.a. TEK), AXL, PDGFRA, and MERTK was involved in the inhibition of cord formation in neo-mode and in the established mode. Thus the anti-angiogenic activity of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is different from ramucirumab and supports the combination of these two agents in cancer treatment to provide further treatment benefit.

### Combination of N-(3-fluoro-4-(l-methyl-6-(1H-pyrazol-4-yl)-1H-indazol-5-yloxy)phenyl)-1-(4-fluorophenyl)-6-methyl-2-oxo-1,2-dihydropyridine-3-carboxamide and DC101 in a Mouse Ear Vascular Model Induced by VEGF-A

The mouse ear vascular model for evaluating anti-angiogenic compounds is set up according to Nagy et al. Methods Enzymol. 2008; 444:43-64. Blood vessels are induced in the mouse ear by VEGF-A (vascular endothelial growth factor A) via the injection of adenoviral vectors carrying the coding sequence of murine VEGF-A into the mouse ears.

DC101 is dosed at 40 mg/kg twice weekly via intraperitoneal injection. N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is dosed at 12 mg/kg orally once daily. For the Day 5 results, the compounds or vehicle control are dosed from days 1-5. For the Day 20 results, the compounds or vehicle control are dosed from days 10-20. For Day 60 results, the compounds or vehicle are dosed from days 50-60. N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide is formulated as a solution in 10% PEG 400 in 90% of 20% Captisol® in H₂O and prepared fresh each week of dosing. DC101 is diluted in PBS each week of dosing. Vehicle control is 10% PEG 400 in 90% of 20% Captisol® in H₂O dosed orally once daily.

The effect of the combination treatment, vehicle control or each single agent was evaluated and quantified with the expression of 50 different vascular markers using the QuantiGene Plex 2.0 Assay kit according to the manufacturer's protocol.

Synergy is determined if the combination is significantly different from control, the effect size is large (combination-control and combination-expected additive response >1.0 or <−1.0), and the p-value for synergy is significant (<0.05). P-values are compared to vehicle control and are Bonferroni adjusted.

Markers that were synergistically affected by the combination treatment were late (day 60) and are markers more for pericytes than endothelium (Table 1). Markers of pericytes are Acta2, Cspg4 (NG2), Notch 1 and Notch 3 and their ligands (DLL1, DLL3, Jag2), and PDGFB. This is consistent in that the combination showed effect in reducing early blood vessels formation and in remodeling early and later blood vessels and in stabilizing normal blood vessels.

TABLE 3
Markers that were affected synergistically by the combination

| | | p-value\hat{ } | | | |
|---|---|---|---|---|---|
| | | | | DC101 + | Synergy |
| Gene | Day | DC101 | LY2801653 | LY2801653 | p-value |
| Normalized | | | | | |
| to 3 house- | | | | | |
| keeper genes | | | | | |
| Acta2 | 60 | 1 | 1 | 0.0092 | 0.013 |
| Igfbp3 | 5 | 0.127 | 0.405 | <0.0001 | <0.0001 |
| Mest | 5 | 0.0005 | 0.07 | <0.0001 | 0.003 |
| Normalized to | | | | | |
| PECAM-1 | | | | | |
| Cdh6 | 60 | 1 | 1 | 0.0009 | 0.013 |
| Cspg4 (NG2) | 60 | 0.21 | 0.11 | <0.0001 | 0.002 |
| DLL1 | 5 | 1 | 0.203 | <0.0001 | 0.008 |
| DLL3 | 60 | 1 | 1 | <0.0001 | 0.003 |
| Gng11 | 60 | 1 | 1 | 0.0007 | 0.001 |
| Hey2 | 60 | 1 | 1 | 0.003 | 0.007 |
| Jag2 | 60 | 0.065 | 0.08 | <0.0001 | 0.028 |
| Mlana | 60 | 0.74 | 1 | <0.0001 | 0.003 |
| Notch1 | 60 | 0.04 | 0.24 | <0.0001 | 0.006 |
| Notch3 | 60 | 0.19 | 0.21 | <0.0001 | 0.004 |
| PDGFA | 60 | 1 | 1 | <0.0001 | 0.003 |
| PDGFb | 60 | 1 | 1 | <0.0001 | 0.001 |
| Ptgds | 60 | 0.35 | 0.68 | <0.0001 | 0.037 |
| Sdc4 | 60 | 0.016 | 0.037 | <0.0001 | 0.019 |

\hat{ }P-values are compared to vehicle control and are Bonferroni adjusted.

Additivity is determined if the combination is significantly different from control, the effect size is large (combination-control and combination-expected additive response >1.0 or <−1.0), and one of the single agents is not significantly different from control and the p-value is not significant for the combination comparing to expected additive response. P-values are compared to vehicle control and are Bonferroni adjusted.

Markers that were not synergistically affected and affected additively by the combination were evenly distributed between early (day 5) and late (day 60) (Table 2). These markers were also an even mix of endothelial markers (e.g. CD34, PECAM1, vwf, PDGFRB, PDGFRA, VEGR2 and its ligands VEGFA) and pericyte markers (e.g. Acta2, Cspg4 (NG2), Notch 1 and Notch 3 and their ligands (DLL1, DLL3, Jag2)). These are consistent with the combination effect throughout the entire study period of days 5-60.

The combination of ramucirumab and N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide resulted in synergistic anti-angiogenic activity in vivo in a mouse ear VEGF-driven angiogenesis model.

TABLE 4
Effect on Markers that were additive by the combination

| | | p-value\hat{ } | | |
|---|---|---|---|---|
| | | | | DC101 + |
| Gene | Day | DC101 | LY2801653 | LY2801653 |
| Normalized to 3 | | | | |
| housekeeper genes | | | | |
| CD34 | 60 | 0.29 | 0.42 | <0.0001 |
| Cdh5 | 5 | 0.31 | 0.03 | <0.0001 |
| Cytl1 | 5 | 0.06 | 0.06 | 0.0001 |
| DLL4 | 5 | 0.23 | 0.97 | 0.0016 |
| DLL4 | 60 | 0.005 | 1 | <0.0001 |
| EphA1 | 60 | 0.03 | 0.23 | 0.0013 |
| Gng11 | 5 | 0.22 | 0.17 | <0.0001 |
| VEGFR2 | 5 | 0.35 | 0.51 | <0.0001 |
| Lor | 60 | 0.25 | 1 | 0.031 |
| Mest | 60 | 0.025 | 1 | 0.005 |
| Notch4 | 5 | 0.07 | 1 | <0.0001 |
| Pdgfd | 60 | 0.013 | 0.34 | 0.0003 |
| PECAM1 | 60 | 0.008 | 0.18 | <0.0001 |
| Rgs5 | 60 | <0.0001 | 0.15 | <0.0001 |
| VEGFc | 60 | 0.74 | 1 | 0.011 |
| Vwf | 5 | 0.78 | 0.35 | 0.0003 |
| Normalized to PECAM-1 | | | | |
| Acan | 5 | 0.03 | 1 | <0.0001 |
| Acan | 60 | 0.04 | 0.69 | <0.0001 |
| Aqp1 | 60 | 0.04 | 0.3 | <0.0001 |
| BMP4 | 60 | 0.02 | 0.64 | <0.0001 |
| Ccl24 | 5 | 0.008 | 0.37 | <0.0001 |
| Cdh6 | 5 | 0.07 | 0.41 | 0.002 |
| Col6a2 | 5 | 0.08 | 0.06 | <0.0001 |
| Col6a2 | 60 | 0.16 | 0.77 | <0.0001 |
| DLL1 | 60 | 0.17 | 0.18 | <0.0001 |
| Dpp4 | 60 | 0.02 | 0.63 | 0.0001 |
| Dtx1 | 5 | 0.51 | 0.001 | <0.0001 |
| Eif3f | 60 | 0.008 | 0.15 | <0.0001 |
| Jag1 | 5 | 0.25 | 0.24 | <0.0001 |
| Jag1 | 60 | 0.04 | 0.08 | <0.0001 |
| Jag2 | 5 | 0.033 | 0.064 | <0.0001 |
| Lef1 | 60 | 0.16 | 0.26 | <0.0001 |
| Lor | 60 | 0.024 | 0.32 | <0.0001 |
| Mest | 5 | 0.0009 | 1 | <0.0001 |
| Mlana | 5 | 0.003 | 0.06 | <0.0001 |
| PDGFa | 5 | 0.019 | 0.06 | <0.0001 |
| PDGFRa | 5 | 0.43 | 0.004 | <0.0001 |
| PDGFRb | 60 | 1 | 1 | 0.003 |
| Sdc4 | 5 | 0.018 | 0.09 | <0.0001 |
| Sp6 | 60 | 0.007 | 0.21 | <0.0001 |
| Tbp | 60 | 0.11 | 0.83 | <0.0001 |
| VEGFa | 5 | 0.19 | 0.75 | <0.0001 |
| VEGFa | 60 | 0.004 | 0.25 | <0.0001 |
| Vwf | 5 | 0.97 | 1 | 0.02 |
| Vwf | 60 | 0.06 | 0.07 | <0.0001 |

\hat{ }p-values are compared to vehicle control and are Bonferroni adjusted

SEQUENCE LISTING

SEQ ID NO: 1
DIQMTQSPSSVSASIGDRVTITCRASQGIDNWLGWYQQKPGKAPKLLIYD
ASNLDTGVPSRFSGSGSGTYFTLTISSLQAEDFAVYFCQQAKAFPPTFGG
GTKVDIK
SEQ ID NO: 2
EVQLVQSGGGLVKPGGSLRLSCAASGFTFSSYSMNWVRQAPGKGLEWVSS
ISSSSSYIYYADSVKGRFTISRDNAKNSLYLQMNSLRAEDTAVYYCARVT
DAFDIWGQGTMVTVSS
SEQ ID NO: 3
DIQMTQSPSSVSASIGDRVTITCRASQGIDNWLGWYQQKPGKAPKLLIYD
ASNLDTGVPSRFSGSGSGTYFTLTISSLQAEDFAVYFCQQAKAFPPTFGG
GTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 4
EVQLVQSGGGLVKPGGSLRLSCAASGFTFSSYSMNWVRQAPGKGLEWVSS
ISSSSSYIYYADSVKGRFTISRDNAKNSLYLQMNSLRAEDTAVYYCARVT
DAFDIWGQGTMVTVSSASTKGPSVLPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYRSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

We claim:

1. A method of treating gastric cancer in a patient, comprising administering to a gastric cancer patient in need of such treatment an effective amount of an antibody comprising a light chain variable region (LCVR) having the amino acid sequence of SEQ ID NO: 1 and a heavy chain variable region (HCVR) having the amino acid sequence of SEQ ID NO: 2, and a compound of the formula: 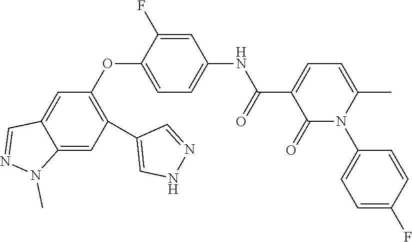 [SMILES: Cc1ccc(C(=O)Nc2ccc(Oc3cc4cnn(C)c4cc3-c3cn[nH]c3)c(F)c2)c(=O)n1-c1ccc(F)cc1]
or a pharmaceutically acceptable salt thereof.

2. The method of claim 1, wherein the compound is N-(3-fluoro-4-(1-methyl-6-(1H-pyrazol-4-yl)-1H-indazol-5-yloxy)phenyl)-1-(4-fluorophenyl)-6-methyl 2-oxo-1,2-dihydropyridine-3-carboxamide.

3. (canceled)

4. The method of claim 1, wherein the antibody is ramucirumab.

5. The method of claim 1, wherein the compound or salt thereof is administered at a dose of about 80 mg/day to about 120 mg/day.

6. The method of claim 4, wherein ramucirumab is administered once every three weeks at a dose of about 6 mg/kg to about 12 mg/kg.

7. A kit comprising an antibody comprising a light chain variable region (LCVR) having the amino acid sequence of SEQ ID NO: 1, and a heavy chain variable region (HCVR) having the amino acid sequence of SEQ ID NO: 2, and a compound of the formula: 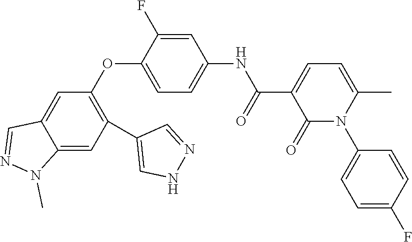 [SMILES: Cc1ccc(C(=O)Nc2ccc(Oc3cc4cnn(C)c4cc3-c3cn[nH]c3)c(F)c2)c(=O)n1-c1ccc(F)cc1]
or a pharmaceutically acceptable salt thereof.

8. (canceled)

9. The kit of claim 7, wherein the antibody is ramucirumab.

10. The kit of claim 7, wherein the compound is N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide.

11. A kit comprising a tablet comprising N-(3-fluoro-4-(1-methyl-6-(1H-pyrazol-4-yl-1H-indazol-5-yloxy)phenyl)-1-(4-fluorophenyl)-6-methyl-2-oxo-1,2-dihydropyridine-3-carboxamide, or a pharmaceutically acceptable salt thereof and a pharmaceutical composition comprising ramucirumab and one or more pharmaceutically acceptable carriers, diluents, or excipients.

12. (canceled)

13. The kit of claim 11, wherein the tablet is formulated by Spray Dried Dispersion.

14. (canceled)

15. (canceled)

16. (canceled)

17. (canceled)

18. (canceled)

19. (canceled)

20. (canceled)

21. (canceled)

22. (canceled)

23. (canceled)

24. The method of claim 4, wherein ramucirumab is administered intravenously or subcutaneously and the compound or pharmaceutically acceptable salt thereof is administered orally.

25. The method of claim 24, wherein the compound or pharmaceutically acceptable salt thereof is formulated into a tablet.

26. The method of claim 25, wherein the tablet comprises a dose of 40 mg of the compound or pharmaceutically acceptable salt thereof.


SEQUENCE LISTING (4 sequences)

SEQ ID NO: 1 - protein, 107 aa, Homo sapiens [sequence omitted]

SEQ ID NO: 2 - protein, 116 aa, Homo sapiens [sequence omitted]

SEQ ID NO: 3 - protein, 214 aa, Homo sapiens
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 446 aa, Homo sapiens
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK